(19) United States
(12) Patent Application Publication
(10) Pub. No.: US 2003/0036749 A1
(43) Pub. Date: Feb. 20, 2003

(54) Method of treating disorders associated with sebaceous follicles

(76) Inventors: Anthony J. Durkin, Costa Mesa, CA (US); Dilip Y. Paithankar, Natick, MA (US); Yacov Domankevitz, Brookline, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/012,241

(22) Filed: Nov. 5, 2001

Related U.S. Application Data
(63) Continuation-in-part of application No. 09/731,496, filed on Dec. 7, 2000.
(60) Provisional application No. 60/170,244, filed on Dec. 10, 1999.

(51) Int. Cl. A61B 18/20

(52) U.S. Cl. 606/3; 606/9

(57) ABSTRACT

Disclosed herein is a method of treating mammalian, for example, human, skin afflicted with a sebaceous follicle disorder, for example, acne. The method involves cooling an exposed surface of a region afflicted with the disorder and applying light, for example, light from a coherent or incoherent light source, to the region. The applied light reduces the size and/or density of lesions associated with the disorder in the treated region, and can reduce or otherwise alleviate lesion-associated skin inflammation in the treated region. Cooling preserves the surface, for example, epidermis, of the skin. The method, therefore, is effective at treating the disorder while at the same time avoiding or minimizing thermal damage to the exposed surface of the skin.

### RELATED APPLICATIONS

This application is a continuation-in-part of co-pending application U.S. Application Ser. No. 09/731,496, filed Dec. 7, 2000, and claims the benefit of U.S. Application No. 60/170,244, filed Dec. 10, 1999, the disclosures of each of which are incorporated by reference herein.

### GOVERNMENT LICENSE RIGHTS

This work was supported, in part, by Federal Grant No. 1-R43-AR 46938-01, awarded under the Small Business Innovation Research Program of the Department of Health and Human Services, Public Health Service. The Government may have certain rights in the invention.

### FIELD OF THE INVENTION

The invention relates generally to a method of treating a mammalian skin disorder associated with sebaceous follicles. More particularly, the invention relates to a method of treating acne in a mammal using a beam of coherent or incoherent radiation.

### BACKGROUND OF THE INVENTION

There are a variety of disorders associated with sebaceous follicles (also referred to herein as sebaceous follicle disorders) known to afflict mammals, in particular, humans. The disorders usually are associated with aberrations (for example, structural or functional aberrations) of the sebaceous follicles. In humans, sebaceous follicles, although present over most of the body surface, usually are largest and most dense on the face, chest and upper back. Accordingly, sebaceous follicle disorders predominantly affect these areas of the human body.

Probably the most pervasive sebaceous follicle disorder in the United States is acne, which affects between 40 to 50 million individuals in the United States (White GM, (1998) “Recent findings in the epidemiologic evidence, classification, and subtypes of acne vulgaris,” J. AM. ACAD. DERMATOL. 39(2 Pt 3): S34-7). Acne occurs with greatest frequency in individuals between the ages of 15 and 18 years, but may begin at virtually any age and can persist into adulthood. In the 12- to 17-year old range, the incidence has been reported to be 25% (Strauss JS, (1982) “Skin care and incidence of skin disease in adolescence,” CURR. MED. RES. OPIN. 7(Suppl 2):33-45). Acne is a disorder characterized by inflammatory, follicular, papular and/or pustular eruptions involving the sebaceous follicles (Stedman's Medical Dictionary, 26^{th} edition, (1995) Williams & Wilkins). Although there are a variety of disorders that fall within the acne family, for example, acne conglobata, acne rosacea, and acne vulgaris, acne vulgaris probably is the most notable and commonly known form of acne. Because acne vulgaris can lead to permanent scarring, for example, facial scarring, this form of acne can have profound and long-lasting psychological effects on an afflicted individual. Furthermore, pustule formation and scarring can occur at an age when the potential impact on an individual is greatest. As a result, enormous amounts of money (i.e., on the order of billions of dollars) are spent annually in the United States on various topical and systemic acne treatments. These treatments often are employed without the guidance or supervision of a physician.

Acne vulgaris typically results from a blockage of the opening of the sebaceous follicle. It is believed that both (i) the amount of sebum, a lipid, keratin and cellular debris containing fluid, produced and secreted by the sebaceous glands and (ii) bacteria, namely, Propionibacterium acnes (P. acnes) which metabolize lipids in the sebum, play a role in formation and development of acne vulgaris. The basic lesion of acne vulgaris is referred to as a comedo, a distension of the sebaceous follicle caused by sebum and keratinous debris. Formation of a comedo usually begins with defective keratinization of the follicular duct, resulting in abnormally adherent epithelial cells and plugging of the duct. When sebum production continues unabated, the plugged follicular duct distends. A blackhead (or open comedo) occurs when a plug comprising a melanin containing blackened mass of epithelial debris pushes up to opening of the follicular duct at the skin surface. A whitehead (or closed comedo) occurs when the follicle opening becomes very tightly closed and the material behind the closure ruptures the follicle causing a low-grade dermal inflammatory reaction. Accordingly, some comedones, for example, in acne vulgaris, evolve into inflammatory papules, pustules, nodules, or chronic granulomatous lesions. Proliferation of P. acnes can result in the production of inflammatory compounds, eventually resulting in neutrophil chemotaxis (Skyes and Webster (1994) DRUGS 48: 59-70).

At present, acne patients may receive years of chronic topical or systemic treatments. Current treatment options include, for example, the use of topical anti-inflammatory agents, antibiotics and peeling agents, oral antibiotics, topical and oral retinoids, and hormonal agonists and antagonists. Topical agents include, for example, retinoic acid, benzoyl peroxide, and salicylic acid (Harrison's Principles of Internal Medicine, 14^{th} edition, (1998) Fauci et al., eds. McGraw-Hill). Useful topical antibiotics include, for example, clindamycin, erythromycin, and tetracycline and useful systemic antibiotics include, for example, erythromycin, tetracycline, and sulphanilamides (see, for example, U.S. Pat. Nos. 5,910,493 and 5,674,539). Administration of the systemic retinoid, isotretinion, has demonstrated some success in the treatment of acne (Harrison's Principles of Internal Medicine, 14^{th} edition, (1998) Fauci et al., eds. McGraw-Hill). Studies indicate that this drug decreases sebaceous gland size, decreases the rate of sebum production and/or secretion, and causes ductal epithelial cells to be less adherent, thereby preventing precursor lesions of acne vulgaris (Skyes and Webster (1994) supra). Side-effects, however, include dry mouth and skin, itching, small red spots in the skin, and eye irritation. A significant concern about oral retinoids is their possible teratogenicity (Turkington and Dover (1996) SKIN DEEP: AN A-Z OF SKIN DISORDERS, TREATMENT AND HEALTH FACTS ON FILE, Inc., New York, page 9). In addition, a variety of hormone-related, for example, corticosteroid anti-inflammatory therapies have been developed for the treatment of acne. These therapies can be expensive and most are associated with deleterious systemic or localized side-effects (Strauss (1982) “Skin care and incidence of skin disease in adolescence,” CURR. MED. RES. OPIN. 7(Suppl 2): 33-45).

Because the foregoing therapies generally do not affect the structure and/or function of sebaceous follicles associated with the disease, the treatments remain non-curative. In other words, the disorder may recur after cessation of therapy. The result can be years of chronic therapy, and potential scarring for the patient, and enormous associated health care costs.

In recent years, a variety of laser-based methodologies for treating acne have been developed. The methods generally involve the combination of laser radiation and either an exogenous or endogenous chromophore present in the target tissue so that the laser light is absorbed preferentially in the target tissue causing morphological changes to the sebaceous follicle and/or causing a reduction of sebum production. For example, U.S. Pat. No. 5,817,089 describes a laser-based method for treating acne requiring topical application of a light absorbing chromophore, for example, micron graphite particles dispersed in mineral oil, onto skin needing such treatment. Similarly, U.S. Pat. No. 5,304,170 also describes a laser-based method for treating acne in which target cells contain greater amounts of a light absorbing chromophore, for example, the carotenoid β-carotene, relative to lesser or non-pigmented surrounding cells. In the chromophore based methods it can be difficult to get sufficient chromophore in the target region to elicit selective tissue damage and the method may still damage the outer layers of the skin resulting in scarring.

### SUMMARY OF THE INVENTION

The present invention addresses the foregoing problems and provides a method for treating sebaceous follicle disorders of mammalian skin, for example, human skin. The invention provides a sub-surface treatment method in which the regions of skin dermis containing sebaceous follicles are treated and the overlying regions of the epidermis/dermis and the underlying portions of the dermis are spared from thermal damage. The invention offers numerous advantages over existing treatment protocols. For example, the method provides a long lasting treatment which persists long after treatment has ceased. Furthermore, the method minimizes trauma and scar formation at the skin surface, reduces side-effects, such as, pain, erythema, edema, and blistering, which can result from other treatments, and can also minimize pigmentary disturbances of the skin.

In one aspect, the present invention features a method of treating a sebaceous follicle disorder in a preselected region of mammalian skin, the preselected region having at least one lesion characteristic of the disorder disposed therein. The method comprises the steps of (a) cooling an exposed surface of the preselected region of the mammalian skin, and (b) applying light having a wavelength in a range from 0.95 microns to 1.16 microns, from 1.30 microns to 1.65 microns, or from 1.85 to 2.20 microns to the preselected region for a time and in an amount sufficient to ameliorate the lesion disposed within the preselected region. Without wishing to be bound by theory, it is contemplated that amelioration of the lesion can result from the destruction of the sebaceous follicle, structural changes to the sebaceous follicle to reduce the possibility of pore blockage, and/or reduction of sebum production by the sebaceous gland associated with the sebaceous follicle.

In a preferred embodiment, the light is coherent or incoherent light, preferably coherent light. The source of the coherent light can be, for example, a pulsed, scanned, or gated continuous wave (CW) laser.

In a preferred embodiment, the light has a wavelength in the range of from 0.95 microns to 1.16 microns, more preferably 0.97 microns to 1.15 microns, and more preferably 1.00 microns to 1.10 microns. In another preferred embodiment, the light has a wavelength in the range of from 1.30 microns to 1.65 microns, more preferably from 1.32 microns to 1.60 microns, more preferably from 1.37 microns to 1.55 microns, and most preferably from 1.40 microns to 1.50 microns. In another preferred embodiment, the light has a wavelength in the range from 1.85 microns to 2.20 microns, preferably 1.90 microns to 2.15 microns, and most preferably from 1.91 microns to 2.10 microns. The light preferably has a fluence in the range from about 0.1 to about 500 joules per square centimeter, more preferably in the range from about 5 to about 150 joules per square centimeter, and most preferably in the range from about 10 to about 75 joules per square centimeter. Alternatively, the light has a power density in the range of about 1 to about 10,000 watts per square centimeter, and more preferably in the range from about 5 to about 5,000 watts per square centimeter.

During practice of the invention, application of the light (heating) energy can induce thermal changes to the portion of the dermis where sebaceous follicles reside. This heating may result in the destruction of the sebaceous follicle or the sebaceous gland associated with the follicle, cause structural changes in the follicle to reduce the likelihood of blockage and/or reduce the level of sebum production. The cooling step serves to preserve the epidermis and the dermis overlaying the sebaceous gland containing region of the skin thereby reducing side-effects such as pain, erythema, edema, and blistering which otherwise may result from exposure to the beam of radiation. The cooling step can be performed prior to, contemporaneous with, or after application of the energy to the target region, or alternatively the cooling can result from a combination of such cooling steps.

Cooling can be achieved using many different techniques known and used in the art. For example, cooling can be achieved by blowing a stream of cold air or gas onto the target site, by applying a cold liquid onto the target site, by conductive cooling using a cold contact surface applied to the target site, or by evaporative cooling using a low boiling point liquid applied to the target tissue. In a preferred embodiment, cooling is achieved using evaporative cooling technologies by means of, for example, a commercially available dynamic cooling device (DCD).

Practice of the invention can be prophylactic or can be performed to ameliorate one or more symptoms or lesions associated with the various sebaceous follicle disorders. Exemplary sebaceous follicle disorders include, for example, acne vulgaris, acne rosacea, acne conglobata, seborrhea, sebaceous adenoma and sebaceous gland hyperplasia. The present invention, however, is particularly useful in the treatment of acne, more specifically, the treatment of acne vulgaris.

Sebaceous follicle disorders, for example, acne vulgaris and seborrhea, sometimes are associated with the overproduction of sebum. For example, in acne vulgaris, the level of sebum production by sebaceous glands has been correlated with the severity of the disorder (Leyden (1995) J. AM. ACAD. DERM. 32: S15-25). Accordingly, in a preferred embodiment, the method of the invention lowers or even eliminates sebum production by sebaceous glands of sebaceous follicles relative to untreated sebaceous follicles. In another embodiment, treatment can increase the size of the opening of the sebaceous follicle, in the proximity of the infundibulum, thereby affecting sebum flow and/or minimizing the likelihood of blockage of the sebaceous follicle. Furthermore, treatment may destroy or inactivate the sebaceous follicle thereby eliminating sebum production in that follicle.

The treatment can reduce the size of one or more lesions, for example, comedones in the case of acne vulgaris, disposed within the preselected region. Furthermore, the treatment can also reduce the number or density of the lesions disposed within the preselected region. In cases in which skin inflammation can be associated with the lesion, for example, in severe cases of acne vulgaris and acne conglobata, the treatment may reduce inflammation associated with the lesion. The benefit of treatment, for example, reduction in the number of or elimination of skin lesions, may become apparent days to weeks after the treatment. Furthermore, it is contemplated that in certain cases, e.g., severe cases, of sebaceous follicle disorders, multiple rounds of treatment, for example, two, three, four, five, six, seven, eight, nine, ten, or more separate rounds of treatment, may be required to treat an individual satisfactorily.

It is contemplated that, based upon choice of appropriate cooling and/or heat energy parameters, it is possible to create thermally induced changes of sebaceous follicles in the absence of an exogenous energy absorbing material. However, under some circumstances, optimal treatment may be facilitated by applying to the preselected region prior to exposure to the radiation beam a light absorbing material, for example, a chromophore photoexcited by the radiation. The radiation absorbing material may be administered systemically to the mammal or applied topically to the preselected region prior to exposure to the radiation beam.

### BRIEF DESCRIPTION OF THE DRAWINGS

The foregoing and other objects, features and advantages of the invention will become apparent from the following description of preferred embodiments of the invention, as illustrated in the accompanying drawings. Like referenced elements identify common features in corresponding drawings. The drawings are not necessarily to scale, with emphasis instead being placed on illustrating the principles of the present invention, in which:

FIG. 1 is a schematic representation of a vertical cross section of a sebaceous follicle disposed within mammalian skin;

FIG. 2 is a schematic representation of an apparatus including a radiation source and delivery system useful in the practice of the invention;

FIG. 3 is a schematic representation of an exemplary hand set of a delivery system in which a beam of coherent radiation and cryogen spray are applied to the same region of the skin surface;

FIG. 4 is a schematic representation of an exemplary timing diagram showing exemplary heating and cooling phases useful in the practice of the invention.

FIG. 5 is a plot showing a profile of temperature (° C.) versus depth through skin (microns) resulting from a first set of exemplary heating and cooling phases; and

FIG. 6 is a plot showing a profile of tissue damage (Omega a.u.) versus depth through skin (microns) resulting from a first set of exemplary heating and cooling phases.

FIG. 7 is a plot showing a profile of tissue damage (Omega a.u.) versus depth through skin (microns) resulting from a second set of exemplary heating and cooling phases.

FIGS. 8A and 8B are bar charts showing the number of acne lesions in regions of patient's skin exposed either to a treatment regime of heating and cooling (FIG. 8A) or a control regime of cooling alone (FIG. 8B).

### DETAILED DESCRIPTION OF THE INVENTION

The present invention is based, in part, upon the discovery that it is possible to treat sebaceous follicle disorders while at the same time preventing or minimizing damage to skin tissue surrounding sebaceous follicles afflicted with the disorder. In particular, sebaceous follicles and dermal regions containing sebaceous follicles are targeted for heat injury whereas the underlying dermal and overlaying dermal and epidermal regions are protected from thermal injury. The underlying dermal regions are protected from thermal injury because, by selection of appropriate parameters, it is possible to limit the penetration depth of the heating energy applied to the region. Accordingly, by choice of appropriate parameters it is possible to heat skin tissue to a preselected depth thereby sparing the underlying tissue from thermal injury. The overlaying dermal and epidermal regions are protected from thermal injury by appropriate surface cooling. Accordingly, by choice of appropriate heating and cooling parameters it is possible for the skilled artisan to induce thermal injury to a specific target zone within the dermis of the skin.

The method of the invention comprises two steps. In one step, an exposed surface of a preselected region of mammalian skin having at least one lesion characteristic of a sebaceous follicle disorder is cooled. In a second step, heating energy in the form of light is applied to the preselected region. The heating energy is applied in an amount and for a time sufficient to induce thermal damage to a portion of the skin containing a sebaceous follicle thereby to reduce or eliminate the production of sebum in the sebaceous follicle or to alter the structure of the sebaceous follicle, for example, by increasing the internal diameter of the follicle, to minimize the possibility of blockage of the follicle. As a result, the treatment ameliorates one or more skin lesions associated with the sebaceous follicle disorder while at the same time preserving the surface of the skin exposed to the heating energy.

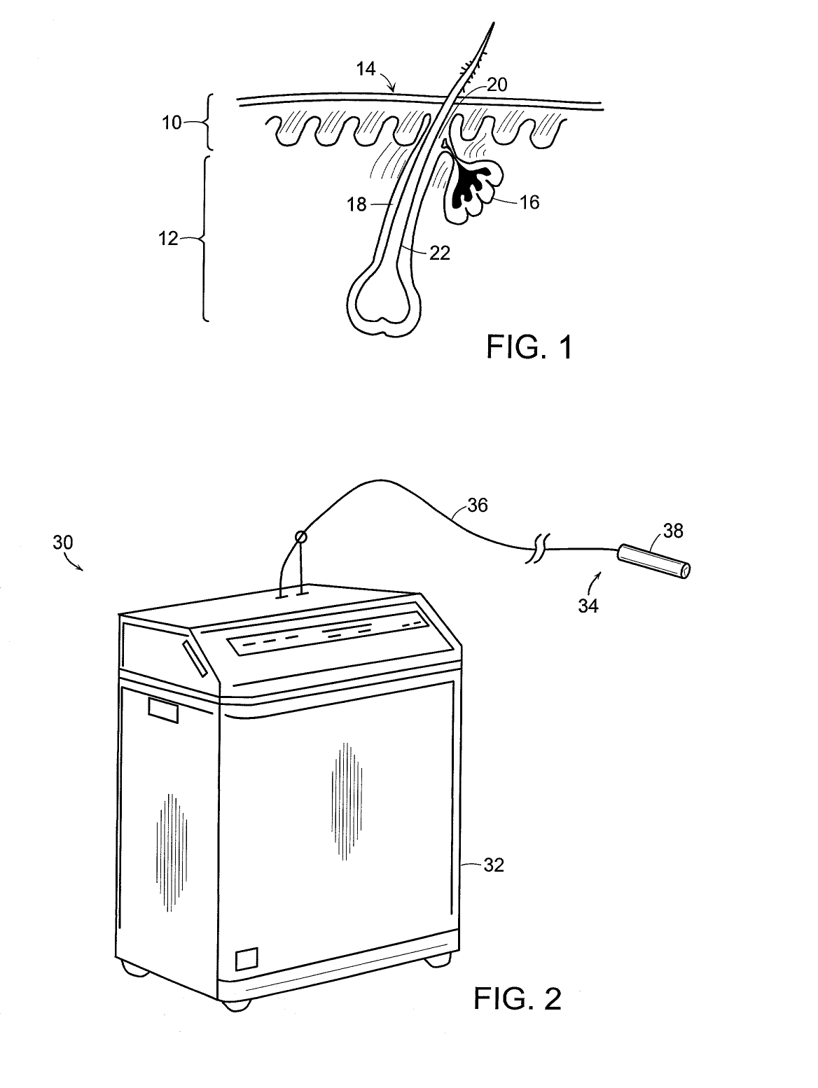

FIG. 1 is a schematic illustration of a cross-sectional view of a sebaceous follicle disposed within human skin. Skin is comprised primarily of two layers in which the top layer of skin, known as the epidermis 10, is supported by a layer known as the dermis 12. The epidermis 10, has an exposed surface 14. In human skin, epidermis 10 extends to a depth of about 60-100 microns from skin surface 14 whereas the underlying dermis 12 extends to a depth of about 4 to 5 millimeters from the skin surface 14. Furthermore, in skin, dermis 12 is supported by or is disposed upon a layer of subcutaneous fat (not shown). Dermis 12 is primarily acellular and comprises primarily water, collagen, and glycosaminoglycans. Water constitutes approximately 60-80 percent of the total weight of the dermis.

As shown, sebaceous gland 16 is in fluid flow communication with a hair duct 18. As a result, sebum produced by the sebaceous gland 16 flows into the hair duct 18. The upper portion of hair duct 18 which receives sebum from sebaceous gland 16 is referred to as the infundibulum 20. Hair shaft 22 is disposed within hair duct 18 and extends beyond the surface of the skin 14. Sebaceous glands usually are located at depths ranging from about 200 to about 1000 microns from the skin surface (Conontagna et al. (1992) in “ATLAS OF NORMAL HUMAN SKIN” by Springer Verlag, New York, N.Y.).

At birth, sebaceous follicles typically contain a small hair, a follicular orifice lined with epithelial cells, and a sebaceous gland. The outer layer of the sebaceous gland lobule is composed of undifferentiated hormonally responsive cells. In response to androgens, these cells, called sebocytes, divide and differentiate. Lipids accumulate and the cells enlarge and rupture, releasing their contents into the hair duct. Sebum, the product of the sebaceous gland, is composed of lipids and cellular debris combined with keratin and microorganisms, including the bacterium P. acnes (Sykes and Webster (1994) supra). Sebaceous glands and the sebum they produce have no proven function in humans, and in fact the skin of young children does not appear to be negatively affected by the almost lack of sebum (Staruss et al. (1992), J. INVEST. DERM., 67:90-97, and Stewart, M. E., (1992) SEMINAR. DERM. 11, 100-105).

As used herein, the term “sebaceous follicle” refers to any structure disposed within mammalian, particularly, human, skin, which comprises a hair follicle, also referred to herein as a hair duct, attached to and in fluid flow communication with a sebaceous gland. As a result, sebum produced by the sebaceous gland flows into the hair follicle. The sebaceous follicle optionally may include a hair shaft disposed within the hair follicle. The upper portion of the hair follicle into which sebum is released from the sebaceous gland is referred to as the infundibulum.

As used herein, the term “sebaceous follicle disorder” refers to any disorder of mammalian skin, in particular, human skin, that is associated with a sebaceous follicle. Sebaceous follicle disorders can result from an over production of sebum by a sebaceous gland of a sebaceous follicle and/or reduction or blockage of sebum flow in the infundibulum of the sebaceous follicle. Exemplary sebaceous gland disorders include, for example, acne, for example, acne vulgaris, acne rosacea, and acne conglobata, seborrhea, sebaceous adenoma and sebaceous gland hyperplasia As used herein, the term “lesion characteristic of the disorder” refers to any skin lesion associated with the sebaceous follicle disorder. For example, lesions associated with acne may include, without limitation, papules and pustules, and skin inflammation associated with the papules and pustules. In addition, specific lesions of acne conglobata include cystic lesions, abscesses and communicating sinuses, whereas specific lesions of acne vulgaris include comedones, cysts, papules and pustules on an inflammatory base. Lesions associated with seborrhea include, without limitation, dermatitis and eczema.

As used herein, the term “ameliorate a lesion” refers to a decrease in the size of a sebaceous follicle disorder-associated lesion and/or density of sebaceous follicle disorder-associated lesions in a preselected region, and can also include a decrease in skin-inflammation associated with the sebaceous follicle disorder.

As used herein, the terms “thermal change” or “thermal injury” with reference to sebaceous follicles refers to any change, for example, structural change and/or functional change, to the sebaceous follicle which ameliorates one or more lesions associated with the sebaceous gland disorder. For example, sebum over-production can be a factor associated with certain sebaceous follicle disorders. Accordingly, practice of the method of the invention can reduce sebaceous gland size and/or sebum production in the area afflicted with the disorder. Reduction in sebum production can occur when sebum producing cells disposed within the sebaceous glands are destroyed and thus inactivated, or when their sebum producing activity is reduced. Furthermore, practice of the method of the invention may result in morphological changes to the sebaceous follicle, for example, increasing the diameter of the follicle, to minimize the likelihood of plug formation. Accordingly, in this type of situation it is possible that, by enlarging the size of the follicle, the chance of plug formation is reduced so that any sebum produced by the sebaceous gland can still flow out of the sebaceous follicle. The changes are thermally induced and may result from the temperature-induced cell death and/or protein denaturation. Accordingly, an objective of the method is to elevate the temperature of the dermal region containing sebaceous glands and more specifically the sebaceous gland to a level and for a time sufficient to cause cell death and/or protein denaturation.

A variety of methods useful in measuring sebum production and useful in the practice of the invention are thoroughly documented in the art. For example, the level of sebum production can be measured by using commercially available sebutape or by means of a sebumeter.

Sebutape is a microporous patch available from CeDerm Corporation (17430 Campbell Rd., Dallas, Texs. 75252). Sebutape detects sebum production without the use of any solvents, powders, or chemicals. The microporous patch acts as a passive collector of sebum. Gradual displacement of air in the pores of the patch changes the patches appearance. The sebum filled pores in the patch do not scatter light and thus appear transparent. The size of the transparent area is a measure of the amount of sebum collected. Patches can be placed on a dark background storage card for evaluation by eye or by computer imaging (Elsner (1995) in “BIOENGINEERING OF THE SKIN: METHODS AND INSTRUMENTATION,” Berardesca, et al., eds., 81-89, CRC Press, Boca Raton, Fla.).

In addition to sebutape, sebum production can be measured by means of a device referred to in the art as a sebumeter, for example, a model SM 810 PC sebumeter, available from Courage & Khazaka (Mathias-Bruggen Str. 91, Koln, Germany). A sebumeter measures the content of sebum in the stratum comeum of skin, the values of which are expressed in micrograms/cm². The sebumeter can be fitted with a manual data collector which has a band designed to absorb skin sebum. The band is 0.1 mm thick and has a 64 mm² contact surface. The higher the amount of lipids present in the band, the higher the film transparency. The numeric values shown on the display are directly proportional to the band transparency and thereby to the amount of lipids present in the band itself (Elsner (1995) supra and http://www.corage-khazaka.de/products.htm and Clarys and Barel (1995) Quantitative Evaluation of Skin Surface Lipids, CLINICS IN DERMATOLOGY 13: 307-321).

Heating of the dermal region may be accomplished by applying to the skin any light source capable of heating living tissue to a depth where sebaceous follicles are located. Heating energy can be provided by coherent light or incoherent light. Coherent light sources, however, are preferred. Coherent light sources useful in the practice of the invention include, but are not limited to, pulsed, scanned or gated CW lasers.

FIG. 2 is an illustration of a system 30 useful in the practice of the invention. The system 30 includes a light source 32 and a delivery system 34. A beam of light generated by the light source 32 is directed to a target region of the individuals skin afflicted with the sebaceous follicle disorder via delivery system 34. The delivery system 34 comprises a fiber 36 having a circular cross-section and a hand piece 38. The light beam having a circular cross-section is delivered by fiber 36 to the hand piece 38. An optical system within the handpiece 38 projects an output beam of light to the target region of the skin. A user holding handpiece 38 can irradiate the target region of the skin with the output beam. In a preferred embodiment, light source 32 is a laser that can produce a beam of pulsed, scanned or gated CW laser radiation. With regard to the light beam, it is contemplated that the wavelength of the beam may be optimized by routine experimentation to maximize absorption by the sebaceous glands and or by the dermis layer of skin where sebaceous glands typically reside (i.e., from about 200 to about 1000 microns from the skin surface).

In another embodiment, the light beam used to thermally injure the sebaceous glands and/or the dermal tissue can originate from a compact, handheld device consisting of a diode laser alone or in combination with additional apparatus such as an optical fiber, doped in such a way so as to delivery energy at a wavelength and power level so as to be therapeutically effective.

The parameter ranges for the beam optimally are selected to cause thermal injury to the sebaceous glands and/or to portions of the dermis where the sebaceous glands typically are present while at the same time avoiding injury to the epidermis and surrounding dermal regions. In particular, the wavelength of the radiation beam can be chosen to maximize absorption by the targeted region of the dermis, and the fluence or power density, depending on the type of radiation, chosen to minimize treatment related side-effects, including, for example, erythema, hypopigmentation, hyperpigmentation, and/or edema.

In order to target regions of the skin containing sebaceous follicles it is desirable to use light that can penetrate the skin to depths in the range of values from about 100 microns to about 2000 microns. It is understood that the depth of penetration of light of a given wavelength depends on the absorption and scattering properties of a tissue of interest. In the visible to near infra-red region of the electromagnetic spectrum, absorption by hemoglobin and melanin contribute to the absorption properties of tissue, whereas in the mid infra-red and far infra-red regions of the electromagnetic spectrum, absorption by water contributes significantly to the absorption properties of tissue. In the mid and far infra-red regions of the electromagnetic spectrum, the light provided preferably has a wavelength that has a water absorption coefficient value preferably in the range of 1 to about 50 cm⁻¹. By choice of appropriate wavelengths it is possible to target selected zones within the dermis of the skin. Provided below are approximate penetration depths of light having different wavelengths, as estimated using two different algorithms.

Table 1 lists wavelength in nanometers versus appropriate penetration depth (δ) in micrometers estimated using the formula:

δ(λ)=1/μₜᵣ(λ)

wherein μₜᵣ(λ) is given by the formula,

μₜᵣ(λ)=μₐ(λ)+μₛ′(λ)

wherein μₜᵣ(λ) id the wavelength dependent total attenuation coefficient, μₐ(λ) is the absorption coefficient, and μₛ′(λ) is the reduced scattering coefficient defined as,

μₛ′(λ)=μₛ(λ)*(1−g(λ))

wherein μₛ(λ) is the signal scattering coefficient and g (λ) is the scattering anisotropy factor.

Values of μₐ(λ) and μₛ′(λ) were taken from Simpson et al. (1998) PHYS. MED. BIOL. 43(9):2465-78 and from measurements of water absorption for estimated typical skin hydration levels of between 60% and 80%. The numbers provided in Table 1 are estimates based upon use of the algorithm and assumptions outlined above. These numbers are meant to provide general guidance and it is understood that the values may vary depending upon the particular type of algorithm and assumptions being relied upon.

TABLE 1

| | Penetration Depth |
|---|---|
| Wavelength (nm) | (microns) |
| 600 | 317 |
| 650 | 339 |
| 700 | 391 |
| 750 | 437 |
| 800 | 487 |
| 850 | 530 |
| 900 | 572 |
| 950 | 602 |
| 1000 | 624 |
| 1320 | 888 |
| 1330 | 867 |
| 1450 | 326 |
| 1550 | 581 |
| 1600 | 681 |
| 1700 | 731 |
| 1800 | 622 |
| 1900 | 133 |
| 2000 | 178 |
| 2100 | 346 |
| 2200 | 440 |
| 2300 | 375 |
| 2380 | 263 |

Similarly, it is possible to estimate approximate penetration depth as a reciprocal of the effective attenuation coefficient, μ_{eff}, calculated from the following equation derived by the diffusion approximation as previously described (Diffusion theory of light transport, section 6.4.1.2, in Optical-Thermal Response of Laser-Irradiated Tissue, (1998) Star, W., eds. Welch, A. J. and van Gemert, M. J. C., Plenum Press, New York):

μ_{eff}={3μₐ[μₐ+(1−g)μₛ]}^{½}, where

μₐ is the absorption coefficient,

μₛ, is the scattering coefficient, and

g is the anisotropy factor.

The typical scattering coefficient and the anisotropy factors in the mid infra-red region have been reported to be 100 cm⁻¹ and 0.9, respectively (Lask G. P. et al. “Nonablative laser treatment of facial rhytides,” Proc. SPIE, 2970, p. 338-349, 1997). These values are approximations. Furthermore, the absorption of skin is assumed to be 70% of the value of the water absorption coefficient. Table 2 lists wavelength in nanometers versus approximate penetration depth (δ) in micrometers using this formula.

TABLE 2

| | Penetration Depth |
|---|---|
| Wavelength (nm) | (microns) |
| 1320 | 1533 |
| 1330 | 1370 |
| 1450 | 230 |
| 1550 | 518 |
| 1600 | 696 |
| 1700 | 813 |
| 1800 | 583 |
| 1900 | 83 |
| 2000 | 113 |
| 2100 | 247 |
| 2200 | 339 |
| 2300 | 274 |
| 2380 | 177 |

In one embodiment, the light has a wavelength in the range from 0.95 to 1.16 microns, more preferably from 0.97 to 1.15 microns, and more preferably from 1.00 to 1.10 microns. In another embodiment, the light has a wavelength in the range from 1.30 to 1.65 microns, more preferably from 1.32 to 1.60 microns, from 1.37 to 1.55 microns, and most preferably from 1.40 to 1.50 microns. In another embodiment, the light has a wavelength in the range form 1.85 to 2.38 microns, more preferably from 1.85 to 2.20 microns, more preferably from 1.90 to 2.15 microns, and most preferably from 1.91 to 2.10 microns. In these ranges, the light is absorbed more preferably by water than by fatty tissue in the skin. As a result, light at these wavelengths heats the water rather than the fatty tissue in patient's skin.

Lasers which produce radiation having wavelengths in the range to be useful in the practice of the invention include, for example, a 1.06 micron Nd:YAG laser, a 1.15 micron helium neon laser, a 1.33 micron Nd:YAG laser, a 1.39 micron Raman shifted Nd:YAG laser, a 1.45 micron diode laser, a 1.48 micron diode laser, a 1.54 micron Er:Glass laser, a 1.54 micron Raman shifted Nd:YAG laser, a 1.57 micron Nd:YAG laser, a 1.91 micron Raman shifted Nd:YAG laser, a 2.10 micron Ho:YAG laser, or another diode laser with appropriate substrate and doping. The light beam may be pulsed, scanned or gated continuous wave laser radiation.

It is contemplated that therapeutically effective dosimitries for coherent sources, for example, pulsed sources, can range from about 0.1 to about 500 joules per square centimeter, more preferably in the range from about 5 to about 150 joules per square centimeter, and most preferably in the range from about 10 to about 75 joules per square centimeter. Similarly, it is contemplated that therapeutically effective dosimitries for incoherent sources can range from about 1 to about 10,000 watts per square centimeter, more preferably in the range from about 5 to about 5,000 watts per square centimeter.

Minimization of thermal injury to the epidermis and the upper layers of the dermis can be accomplished by cooling the skin surface prior to, contemporaneous with, and/or after heating the sebaceous gland containing portion of the dermis. Furthermore, if the heating source is pulsed, cooling can be applied at intervals between the heating pulses. It is contemplated that the light delivery system also may include an integrated cooling system for cooling the skin surface prior to, contemporaneous with, and/or after the application of the energy beam. Accordingly, such an energy delivery system would be multi-functional, i.e., capable of both delivering an energy beam and cooling the surface of the skin at the same time.

Cooling may be facilitated by one or more cooling systems known and used in the art. Cooling systems useful in the practice of the invention may include, without limitation: blowing a cold stream of gas, for example, cold air, or cold N₂ or He gas, onto the surface of the skin (Sturesson and Andersson-Engels (1996) “Mathematical modelling of dynamic cooling and pre-heating, used to increase the depth of selective damage to blood vessels in laser treatment of port wine stains,” PHYS. MED. BIOL. 41(3):413-28); spraying a cold liquid stream onto the surface of the skin (Sturesson (1996) supra); conductive cooling using a cold contact surface which does not interfere with the method of heating, for example, a cooled transparent optical material, such as a cooled sapphire tip, see, for example, U.S. Pat. No. 5,810,801; applying a low boiling point, non-toxic liquid, for example, tetrafluoroethane or chlorodifluoromethane, onto the surface of the target tissue, to cool the tissue surface by evaporative cooling, or applying a low boiling point non-toxic liquid onto the surface of the target tissue combined with blowing a stream of gas in the vicinity of the liquid to remove at least a portion of the liquid (U.S. patent application No. 20,010,009,997A1, published Jul. 26, 2001).

In a preferred embodiment, cooling is facilitated by a dynamic cooling device (DCD), such as a DCD manufactured by Candela Corporation. Applications of the DCD have been described in the art and include, for example, Anvari et al. (1996) APPLIED OPTICS 35:3314-3319; Anvari et al. (1997) PHYS. MED. BIOL. 42:1-18; Ankara et al. (1995) LASERS IN MEDICAL SCIENCE 10:105-112; and Waldorf et al. (1997) DERMATOL. SURG. 23:657-662, U.S. Pat. Nos. 5,820,626 and 5,814,040 and PCT/US97/03449. The DCD provides a timed spray of fluid onto the surface of the skin, prior to, contemporaneous with, and/or after the application of the energy beam. Unlike steady-state cooling, for example, an ice cube held against the tissue, dynamic cooling primarily reduces the temperature of the most superficial layers of the skin. For example, it has been estimated that the use of tetrafluoroethane as a cryogen may result in a drop in surface-temperature of about 30-40° C. in about 5-100 ms (see, Anvari et al. (1991) supra).

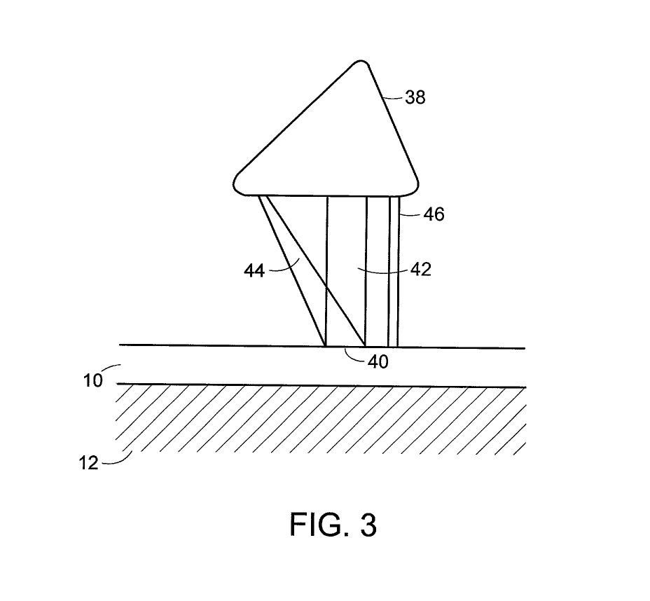

Operation of such an embodiment is shown schematically in FIG. 3. Briefly, hand piece 38 is used to apply a beam of light 42 from a laser source and a cryogen spray 44 to preselected region 40 of the skin surface. Application of the heat energy together with surface cooling cause thermal injury to the sebaceous follicle containing portion of the dermis while preserving epidermis 10. Guide 46 ensures that the handpiece 38 is positioned at the appropriate height above the surface of the skin to ensure that the beam of radiation 42 and the cryogen spray 44 both contact skin surface at the preselected region 40.

The preselected region can be cooled prior to, contemporaneous with, and even after the application of the energy beam. The relative timing of cooling the skin surface and the application of heating energy depends, in part, on the depth to which thermal injury is to be prevented. Longer periods of cooling prior to the application of radiation allow more time for heat to diffuse out of the tissue and cause a thicker layer of tissue to be cooled, as compared to the thickness of the layer cooled by a short period of cooling. This thicker layer of cooled tissue sustains less thermal injury when the heating energy is subsequently applied. Continued cooling of the skin surface during the delivery of heating energy extracts heat from the upper layers of the skin as heat is deposited, thereby further protecting the upper skin layers (e.g., epidermis and dermis overlaying the target region) from thermal injury.

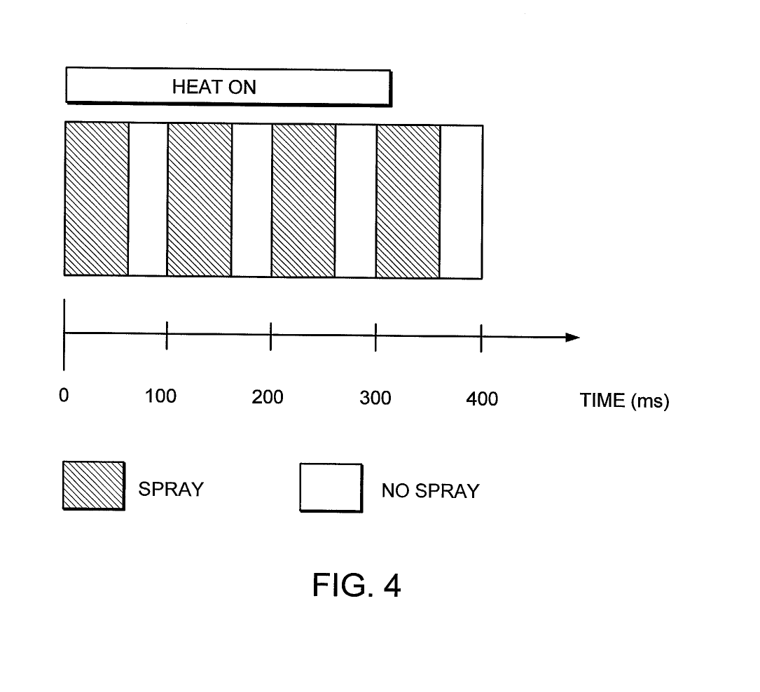

FIG. 4 provides an exemplary timing diagram showing time phases for the heating and/or cooling of the skin tissue afflicted with the disorder. The heating phase, represented by the horizontal bar, has a duration of 300 ms. Cooling, represented by vertical bars, comprises four separate cycles having a duration of 100 ms, each cycle comprising a 70 ms period when cryogen spray is applied to the skin surface and a 30 ms period when no cryogen spray is applied to the skin surface. In this timing diagram, the skin surface is cooled both (i) at the same time (i.e., the 70 ms phases of the first three cooling cycles) as the skin is exposed to the radiation beam and (ii) after (i.e., the 70 ms phase of the fourth cooling cycle) the skin has been exposed to the radiation beam.

Another exemplary timing scheme that may be used in the practice of the invention is similar to the previous scheme except that the light energy is provided intermittently with cooling steps in-between each of the heating steps. For example, an exemplary scheme may include a pre-laser application of coolant, a first laser pulse, an intervening application of coolant, a second laser pulse, an intervening application of coolant, a third laser pulse, an intervening application of coolant, a fourth laser pulse, and finally a post-laser application of coolant. In this type of scheme, the laser pulses can have the same or different durations. In a preferred scheme, the laser fluence ranges from about 8 to 24 J/cm² at a wavelength of 1450 nm. The total laser duration is 210 ms which is divided into four pulses of equal durations with three spurts of cryogen spray interspersed between the four laser pulses. In addition, there is a pre-laser spray and a post-laser spray. A 1450 nm laser and DCD system, Smoothbeam™ is available from Candela Corporation and can be used in the practice of the invention. The laser provides a maximum output power on the skin of 15 W. Using such a device with a pulse duration of 210 ms, a maximum fluence of 25 J/cm² can be achieved with a 4 mm circular spot at a repetition rate of 1 Hz. In order to speed up treatment times, it may be desirable to use laser beams with a spot size greater than 4 mm in diameter. This can be achieved if the power output of the laser is increased.

In another embodiment, the light delivery and cooling systems may comprise separate systems. The cooling system may comprise a container of a cold fluid. Cooling the surface of the skin can be accomplished by applying the cold fluid onto the skin which then extracts heat from the skin on contact. In such an embodiment, a light delivery system comprises, for example, a handpiece containing optics for directing, collimating or focusing the radiation beam onto the targeting region of the skin surface. The light beam can be carried from the energy source, for example, a laser, to the handpiece by, for example, an optically transparent fiber, for example, an optical fiber. Coolant from a separate reservoir can be applied to the surface of the targeted region. In this embodiment, coolant from the reservoir flows to a dispensing unit separate from the energy delivery system via tubing connecting the reservoir and the dispensing unit. The coolant, once dispensed, can be retained in situ on the surface of the targeted region by a ring, for example, a transparent ring, which can be attached to the energy delivery system.

Selective heating of dermal regions containing the sebaceous glands can be achieved by selecting the appropriate heating and cooling parameters. For example, by choosing the appropriate wavelength it is possible to selectively heat portions of the dermis to a desired depth. For example, it is estimated that light having a wavelength of 1000 nm penetrates to a depth of approximately 600 microns. Accordingly, it is contemplated that dermal tissue greater than 600 microns from the skin surface will not be subjected to such intense heating as the region within 600 microns of the skin surface. Furthermore, it is possible to prevent damage to the skin surface by applying the types of cooling discussed hereinabove. By choosing appropriate parameters for the heating and cooling steps it is possible to selectively heat and thus selectively damage particular zones (target regions) within the skin which may contain a sebaceous gland and/or an infundibulum of a sebaceous follicle. Specifically, by choosing the radiation wavelength, the timing of the surface cooling, the cooling temperature, the radiation fluence and/or the power density as described above, the depth, thickness and degree of thermal injury can be confined to a particular zone within the dermis. Optimization of the foregoing parameters can be used to selectively heat regions of the dermis containing sebaceous follicles, more preferably regions containing sebaceous glands, while at the same time substantially or completely sparing injury to overlying regions of epidermis and dermis as well as underlying layers of dermis.

Practice of the method of the invention preferably results in the targeted region of the dermis being heated to a temperature in the range from about 50° C. to about 85° C., and more preferably from about 60° C. to about 70° C. This temperature rise can be sufficient to affect the structure and/or function of sebaceous follicles disposed within the targeted region of the dermis. Studies have indicated that temperatures of 60° C. and above may be sufficient to create thermal damage to skin (Weaver & Stoll (1969) AEROSPACE MED 40: 24). The cooling system on the other hand, preferably cools the area of the skin above the targeted dermal region to temperatures below about 60° C., more preferably to below 50° C. during application of the heating energy, thereby minimizing or avoiding collateral thermal damage to the epidermis.

Although the method of the invention can treat sebaceous follicle disorders in the absence of an exogenously added energy absorbing material, under certain circumstances, it may be beneficial to introduce such a material into the targeted region prior to application of the heat energy. For example, where the energy source is a beam of coherent or incoherent radiation, an externally injected radiation absorber, for example, a non-toxic dye, for example, indocynanine green or methylene blue, can be injected into the targeted dermal region. A radiation source provides radiation which is absorbed by tissue containing the absorber. As a result, use of a radiation absorbing material in combination with surface cooling can confine thermal injury or damage to the targeted dermal regions thereby minimizing potential injury to surrounding tissue.

### EXAMPLES

Practice of the invention will be more fully understood from the following examples, which are presented herein for illustrative purposes only, and should not be construed as limiting the invention in any way.

### Example 1

### Computer Modeling of Treatment Parameters

Mathematical calculations were performed to determine whether certain heating and cooling schemes could produce the desired temperature profiles in tissue suitable for treating sebaceous follicle disorders. Monte Carlo simulations of light transport and finite difference numerical calculations of temperature distribution identified initial heating and cooling parameters for testing in ex vivo and in vivo models.

Specifically, stochastic Monte Carlo simulations of light transport were performed to calculate the distribution of light fluence within a tissue. Given the light distribution and the absorption coefficient, the heat generated by the light was calculated at different depths within the tissue. Numerical finite difference heat transfer calculations taking into account the cooling provided by the cryogen spray were performed to calculate the spatial thermal profiles in tissue at various time points. The temperature profiles are indicative of the tissue damage produced and detailed calculations of thermal damage were done using a kinetic model. Such calculations are a valuable tool in evaluating various heating and cooling schemes to produce desired temperature profiles and can be used as a guide in actual ex vivo or in vivo experiments.

The kinetic thermal damage model relates the temperature-time history of tissue to the thermal damage. The thermal damage measure, Q, is traditionally defined as the logarithm of the ratio of the original concentration of native tissue to the remaining native state tissue and by using a kinetic model, it is given at a time (t) by the formula:

$$Ω ( t ) = ln \{ C ( 0 ) / C ( t ) \} = {\int }_{0}^{t} \{ A exp ( - {E}_{a} / RT ( τ ) ) \} ⅆ τ$$

where A is a pre-exponential factor, Eₐ is the activation energy, R is the Boltzmann constant, and T(τ) is the thermal history as a function of time (Pearce and Thomsen (1995) “Rate process analysis of thermal damage,” in “OPTICAL-THERMAL RESPONSE OF LASER-IRRADIATED TISSUE” Welch and van Gemert, eds., Plenum Press, pp. 561-603). The characteristic behavior of the kinetic damage model is that, below a threshold temperature, the rate of damage accumulation is negligible, and it increases precipitously when this value is exceeded. This behavior is to be expected from the exponential nature of the function. Pearce and Thomsen, supra, define a critical temperature, T_{crit}, as the temperature at which the damage accumulation rate, dΩ/dt is 1.0. Id. This criterion gives T_{crit} as Eₐ/R ln(A). A range of values for T_{crit} from 60° C. to 85° C. has been reported for various human tissue (Pearce and Thomsen (1995) supra). For example, Stoll and Weaver report a critical temperature of 60° C. for human skin (Weaver and Stoll (1969) “Mathematical model of skin exposed to thermal radiation” AEROSPACE MED. 40:24).

Monte Carlo and heat transfer calculations were performed using appropriate scattering and absorption properties for light having a wavelength of 1450 nm (Table 3). Heat transfer calculations were performed numerically by a finite-difference method taking into account the cooling due to the cryogen (tetrafluoroethane, an EPA approved refrigerant) and heating due to the laser absorption by tissue using the parameters set forth in Table 4.

TABLE 3
Optical Properties Used in Monte Carlo Model for Light Distribution

| | Refrac- | | | |
|---|---|---|---|---|
| Property → | tive | Absorption | Scattering | Anisotropy |
| Component ↓ | Index, n | Coefficient, μₐ | Coefficient, μₛ | factor, g |
| Air | 1 | 0 | 0 | 0 |
| Skin | 1.37 | 20 cm⁻¹ | 120 cm⁻¹ | 0.9 |

TABLE 4
Values of Parameters Used in Heat Transfer Calculations

| | | | No. of 100 | Spray | Pre, post- | Cryogen-skin |
|---|---|---|---|---|---|---|
| Optical | Laser | Cryogen | ms cooling | duration | laser spray | heat transfer |
| Power | Duration | Temp. | cycles | per cycle | duration | coefficient |
| 10.5 W | 300 ms | −26° C. | 3 | 50 ms | 30, 30 ms | 5000 W/m²K |

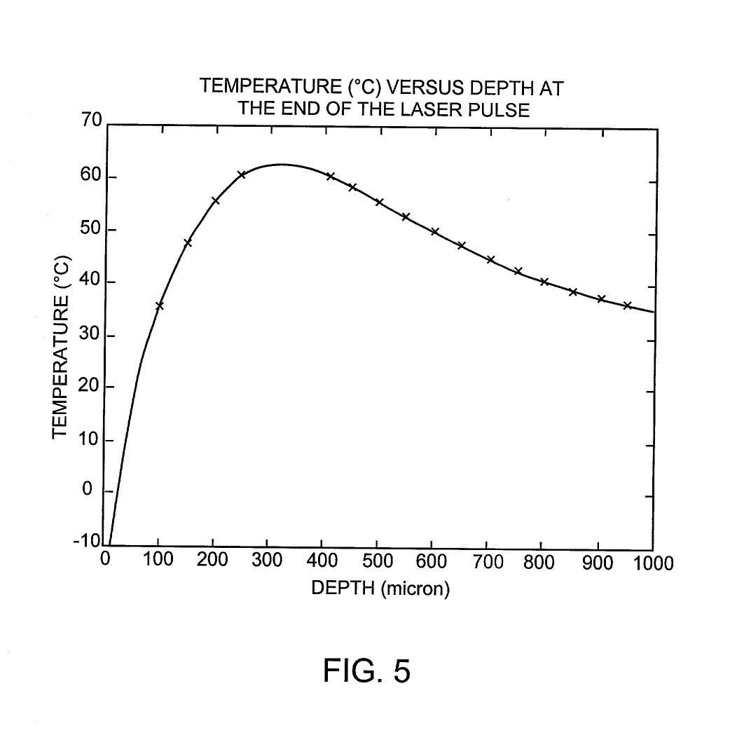

Using the theoretical parameters set forth in Table 4, laser light having a wavelength of 1400 nm was delivered for 300 ms at a power of 10.5 W. Simultaneously with the beginning of the laser, the first of the three cryogen cooling cycles were delivered. Each cooling cycle lasted for 100 ms, each comprising 50 ms of spray and 50 ms of no spray. Such a cooling scheme provides almost constant cooling of the top layer of the skin and is expected to lead to epidermal preservation. Spatial temperature profiles were calculated at various times for a typical set of heating and cooling parameters expected to be effective in treatment. FIG. 5 shows the temperature (° C.) plotted versus depth (microns) at the end of the laser pulse. Since tissue temperature in the dermal band centered at about 300 microns exceeds 60° C., a critical temperature reported for skin (Weaver and Stoll (1969) supra), thermal alteration of tissue is expected in this region of skin.

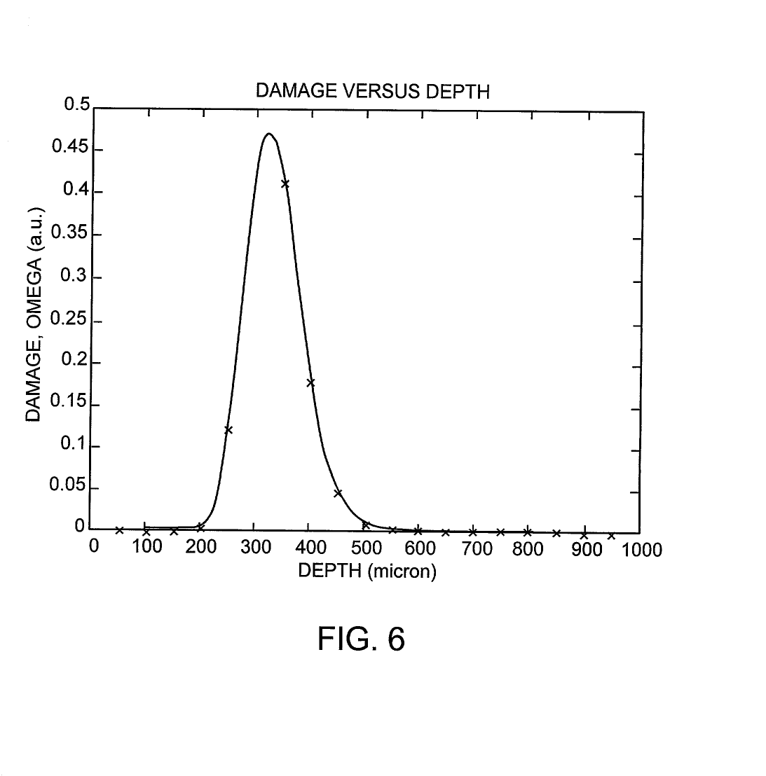

In addition, calculations were performed to determine the extent of tissue damage as a function of depth. Parameters inputted into the kinetic thermal damage model were Eₐ=6.28×10⁵ J/mole and A=3.1E98 s⁻¹ to give a T_{crit} value of 60.1° C. The calculated temperature profile through the center of the treatment area as a function of depth is shown in FIG. 5. The peak temperature occurs at a depth of about 300 microns. FIG. 6 depicts the damage predicted by the kinetic thermal damage model as a function of depth. Although the magnitude of the damage depends strongly on the parameters used in the expression for damage, based on these calculations, it is estimated that a thermal damage band occurs between the depths from about 220 microns to about 450 microns. Because sebaceous glands typically are located from about 200 to about 1000 microns from the skin surface, the zone of thermal damage predicted by the foregoing calculations likely would contain sebaceous glands.

In addition, heat transfer calculations were performed using the optical properties set forth in Table 3 together with a different set of treatment parameters set forth in Table 5.

TABLE 5
Values of Parameters Used in Heat Transfer Calculations

| | | | | | | Thermal | Cryogen- |
|---|---|---|---|---|---|---|---|
| | | | Pre-laser | Intermediate | Post-laser | Diffusivity | skin heat |
| Laser | Laser | Cryogen | spray | Spray | spray | of Tissue, | transfer |
| Fluence | Duration | Temp. | Duration | duration | duration | k/pCₚ | coefficient |
| 10 J/cm² | 210 ms | −44° C. | 10 ms | 30 ms | 20 ms | 8 × 10⁻⁴ cm²/s | 8000 W/m²K |

In the calculations, laser light having a wavelength of 1450 nm was delivered for a total of 210 ms in four separate but equal intervals at a fluence of 10 J/cm². Three cryogen cooling cycles 10 ms in duration were delivered between the four heating steps to give a total spray duration of 30 ms. The timing scheme further included a 10 ms pre-laser spray of coolant, and a 20 ms post-laser spray of coolant. Spatial temperature profiles were calculated at various times for a typical set of heating and cooling parameters expected to be effective in treatment. The epidermis is kept from heating by repeated cryogen pulsing and thermal heating of the upper dermis is achieved. The peak temperature was calculated to be 62° C. at the end of the last laser pulse for the given cryogen regimen and a laser fluence of 10 J/cm².

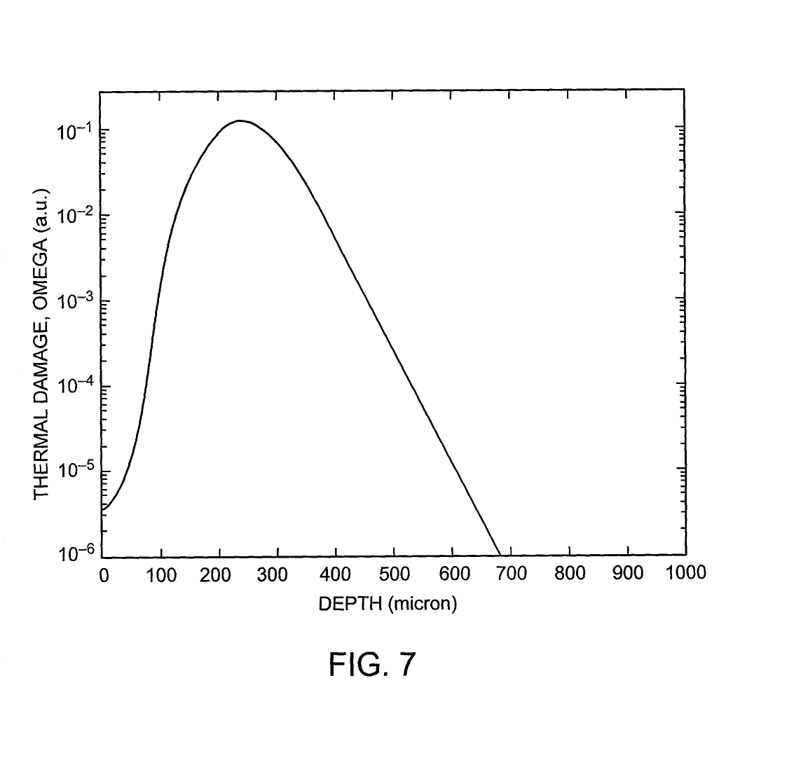

Calculations were also performed to determine the extent of tissue damage as a function of depth. Parameters inputted into the kinetic thermal damage model were Eₐ=6.28×10⁵ J/mole and A=3.1×10⁹⁸5⁻¹ to give a T_{crit} value of 60° C. FIG. 7 shows the predicted damage profile on a log scale as a function of depth. Based on these calculations, it is estimated that a thermal damage band occurs between the depths from about 100 microns to about 600 microns. Using these conditions, maximal thermal damage is estimated to occur about 250 microns from the skin surface. Because sebaceous glands typically are located from about 200 to about 1000 microns from the skin surface, the zone of thermal damage predicted by the foregoing calculations likely would contain sebaceous glands.

Based on the two exemplary sets of parameters set forth in this Example, it is apparent that a variety of heating and cooling schemes can be used in the practice of the invention.

### Example 2

### Ex vivo Pig Skin Study

To assess if it was possible to preserve skin epidermis while damaging the dermis as well as to assess the zone of dermal damage, experiments were performed ex vivo with freshly excised white pig skin samples.

The temperature of the skin sample was maintained at 30° C. by placing the sample on a warm 1 inch teflon pad and by simultaneous heating from the top with a heat lamp. Several spots on the skin were irradiated using different heating and cooling parameters. A spot size of 4 mm was irradiated using a diode laser system having a wavelength of 1.45 microns and with an optical power of 14 W. A scheme for the timing of the cryogen spray was used that provided almost simultaneous cooling of the skin to preserve the epidermis. The heating and cooling were turned on for a time period ranging from 100 ms to 300 ms. Energy fluences at the skin surface as high as 33 J/cm² were used. Immediate post-treatment 4-mm punch biopsies were performed and the biopsy samples fixed in 10% buffered formalin solution. The samples were processed and stained with hemotoxylin and eosin (H&E) stain and analyzed under an optical microscope. Thermally denatured collagen appears purple whereas the non-damaged collagen appears pink with this stain under visual examination. The results are summarized in Table 6.

TABLE 6
Values of Parameters used and Observations

| Laser | Cooling | Energy/ | Epidermis condition | Depth of the band of thermal |
|---|---|---|---|---|
| (ms) | #x (ms + ms) | pulse (J) | (biopsy observation) | damage (estimated by biopsy) |
| 200 | 3x (30 + 70) | 2.82 | epidermis separated | 000 → 500 μm (500 μm) |
| 200 | 3x (40 + 60) | 2.82 | epidermis spared | left cut: 100 → 400 μm (300 μm) |
| | | | | right cut: 200 → 300 μm (100 μm) |
| 200 | 3x (50 + 50) | 2.82 | epidermis spared | 150 → 300 μm (150 μm) |
| 200 | 3x (60 + 40) | 2.82 | epidermis intact | none |
| 200 | 3x (70 + 30) | 2.82 | epidermis intact | none |

In Table 6, the first column provides the total time during which the laser was turned on. The second column provides the cooling parameters. The cooling period was divided into different number of cycles, each lasting 100 ms. Each cooling period having a certain duration when cooling spray was applied and the remainder when no cooling spray was applied. For example, the cooling parameter of 3×(30+70) comprises 300 ms of total cooling with the following timing: (30 ms spray+70 ms no spray)+(30 ms spray+70 ms no spray)+(30 ms spray+70 ms no spray). The last 100 ms cycle is the post-laser spray. The third column provides the total laser energy per pulse. The fourth column provides the epidermal condition as observed by microscopic observation of the biopsy. The fifth column provides the depth of the band of thermal damage as observed in the skin by microscopic observation.

Some notable observations for 200 ms of laser and different cooling parameters are shown in Table 6. With 200 ms of laser at 14W and 3 cycles of cooling, each lasting 100 ms and comprising of 40 ms of spray and 60 ms of no spray, thermal damage was localized to a zone ranging from about 100 to about 400 microns in depth from the skin surface while at the same time preserving the epidermis.

### Example 3

### Human Study

Similar treatment parameters as described in the above pig skin study were used to treat sites behind the ear in a human study. Examination of biopsies taken immediately after the treatment showed that sebaceous glands were damaged while skin epidermis was completely spared.

In a separate study, 4 mm spots at periauricular sites (behind the ear) were irradiated, again, with varying combinations of heating and cooling parameters. Heating was provided by a 12W CW 1.45 micron laser and cooling was provided with a DCD system available from Candela. The heating phase included a single 300 ms exposure to coherent light produced by the 12 W CW 1.45 micron laser. Cooling was accomplished by means of three cooling cycles of 100 ms in duration, with each cooling cycle comprising 20 ms of cryogen spray and 80 ms of no cryogen spray. Two treatments were performed per site.

The results confirmed that it is possible to induce thermal alteration of sebaceous glands extending 200-400 micron in the dermis while preserving the epidermis. Using these parameters, no significant visible epidermal side-effects were detectable. Because this experiment confirms that it is possible to selectively alter sebaceous glands disposed in human tissue, it is contemplated that the parameters employed may also be useful in ameliorating within a preselected region the symptoms, for example, reducing the size and/or density of cysts, papules, pustules, associated with the sebaceous follicle disorder.

### Example 4

### Rat Study

Rat studies may also be used to further characterize and delineate optimal heating and cooling parameters useful in ameliorating lesions associated with a sebaceous follicle disorder prior to initiation of a systemic human trial.

In particular, experiments can be used to demonstrate the (1) alteration of the sebaceous glands and associated structures, (2) epidermal preservation, and (3) effectiveness of different parameter ranges. The aim of the pilot study is to determine if thermal alteration of the sebaceous glands is possible and to determine approximately the effective range of parameters which at the same time minimize side-effects such as blisters and scars.

A laser beam of 1.45 micron wavelength at 14W optical power will be used. The parameters shall span the following range: laser, 50 ms-400 ms; cooling cycle, 100 ms; spray, 20-80 ms per cooling cycle. For example, 2 cycles of 20 ms per cooling involves 20 ms spray+80 ms no spray+20 ms spray+80 ms no spray. The number of cooling cycles will match the laser time. For example, 2 cooling cycles will be used for laser times ranging from 200-290 ms. Additional sprays, each lasting 30 ms, will be employed before and after laser treatment. A preferred set of parameters is 250 ms of laser at 14W, with 30 ms spray/100 ms cooling cycle, and pre-laser and post-laser sprays of 30 ms each.

Histology of biopsies will be used to quantitatively assess the thermal alteration of the sebaceous glands. These results will be used to tune the heating and cooling treatment parameters for the next rat. For example, if the epidermis is not spared, duration of cooling spray will be increased. If the alteration of the sebaceous glands is not large enough, heating times will be increased. It is contemplated that such iterations will give an optimum set of heating and cooling parameters.

Seven white hairless male rats each having reached puberty (ages 7 to 8 weeks) will be used in the initial study. Each rat will be treated and examined one at a time. Data obtained will be used in improving the parameters for further treatment.

Sebutape will be placed on various parts of a first rat for an hour, and the sebum producing areas on the rat skin determined. On the following day, the experiment will be repeated to demonstrate the reproducibility of the sebutape technique for identifying zones of sebum production. Then, the rat will be sacrificed and skin biopsies taken at various sites on the body to map the density of occurrence of sebaceous glands over the back, the belly, and the ears of the rat. The results will be correlated with the results from the sebutape measurements.

The remaining rats will them be treated and alterations to the structure and/or function of the sebaceous glands will be measured. A second rat will be allowed to acclimatize for 3 days. On day zero, six areas for treatment will be delineated on the rat's back with a felt tip pen or tattoo. Each treatment area will be made large enough to provide at least two biopsies. Also, on day zero, a control biopsy will be taken, assuming that there is no large variation in sebaceous glands density over the back as observed with the sebutape and biopsy experiments on the first rat. Also, on day zero, six different marked areas will be treated with six different parameter sets; one set may consist of only cryogen and no laser. Also, on day zero, after two hours, ‘immediate’ post-operative biopsies of all treatment sites will be taken and each wound sutured. Six biopsies will be obtained. On day 1, i.e., 24-hour post-treatment, the animal will be sacrificed by administration of sodium pentobarbital and six necropsies of the treated areas will be obtained.

Histology analysis will include quantification of alteration to sebaceous glands as well as measurement of fibroblasts, fibrocytes, collagen content and type, epithelial cells, and dermal characteristics. H&E and viability stains will also be used. Histological analyses of immediate biopsies and 24-hour necropsies will be used to assess the alteration of the sebaceous glands. These results will be used to tune the heating and cooling treatment parameters for the next rat. Successful treatment shall be estimated when there is a reduction in size or alteration of the sebaceous glands by at least 25%.

### Example 5

### Human Clinical Study

A clinical study has been performed to evaluate the effectiveness of a 1450 nm wavelength laser in conjunction with a DCD for the treatment of acne. Male subjects with acne of similar severity on the upper back were recruited for the study.

The treatment areas received laser and cryogen whereas the control areas received only cryogen spray. The treatment and control sites were mapped on transparent paper to track the location of lesions and ensure the accuracy of site selection and lesion counts at all time points. The areas of treatment and control sites on the back were up to approximately 36 cm². When possible, four separate treatments were provided with each treatment being three weeks after the previous one.

The patients were treated with a 1450 nm diode laser and DCD system available from Candela Corporation. The fluences ranged from about 15 J/cm² to about 22 J/cm² and the cryogen spray was tetrafluoroethane. The timing scheme for each treatment included a 10 ms pulse of cryogen spray, a 52.5 ms laser pulse, a 10 ms pulse of cryogen spray, a 52.5 ms laser pulse, a 10 ms pulse of cryogen spray, a 52.5 ms laser pulse, a 10 ms pulse of cryogen spray, a 52.5 ms laser pulse, and a 20 ms pulse of cryogen spray.

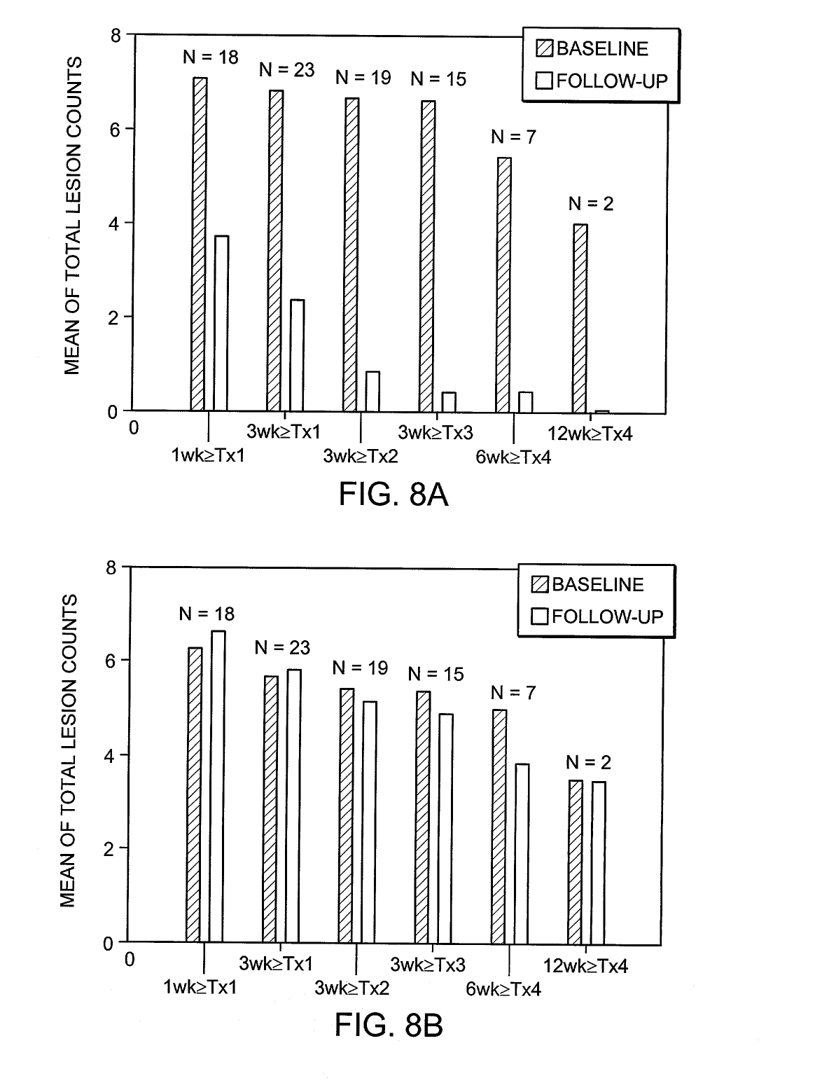

The patients were provided with 1 to 4 separate treatments at intervals of three weeks. The results of the study are summarized in FIGS. 8A and 8B. FIG. 8A represents the numbers of lesions that were counted in individuals from regions having received the treatment regime (i.e., both heating and cooling). FIG. 8B represents the numbers of lesions that were counted in individuals from regions having received a control regime (cooling alone). In both figures, N represents the numbers of patients for which the acne lesions were counted in predetermined regions during follow-up sessions, the filled bars represent the mean number of lesions in the regions of those individuals who attended the follow-up sessions prior to receiving either the first treatment regime (FIG. 8A) or the first control regime (FIG. 8B), and the stippled bars represent the mean number of lesions in the same regions of individuals after receiving either the treatment regime (FIG. 8A) or the control regime (FIG. 8B).

FIGS. 8A and 8B include data derived from six follow-up sessions. The first follow-up was 1 week after treatment (denoted by 1 wk→Tx1). Eighteen people attended the first follow-up. The second follow-up was 3 weeks after treatment 1 (denoted by 3 wk→Tx1). Twenty three people attended the second follow-up. At that time, those individuals were exposed to a second round of treatment and control regimes. A third follow-up was 3 weeks after treatment 2 (denoted by 3 wk→Tx2). Nineteen people attended the third follow-up. At that time, those individuals were exposed to a third round of treatment and control regimes. A fourth follow-up was 3 weeks after treatment 3 (denoted by 3 wk→Tx3). Fifteen people attended the fourth follow-up. At that time, those individuals were exposed to a fourth round of treatment or control regimes. A fifth follow-up was six weeks after the treatment 4 (denoted by 6 w→Tx4). Seven people attended the fifth follow-up. A sixth follow-up was 12 weeks after the treatment 4 (denoted by 12 wk→Tx4). Two people attended the sixth follow-up.

Photographs of the treatment and control sites were taken before the initial treatment and during every visit for treatment or follow-up. Photographs were used for visual assessment of lesion counts as well as to confirm and review the lesion counts at the end of the study by comparison of photographs by blinded observers. Clinical observations of the treatment and control sides were graded and recorded. These observations included new or recurrent lesion counts, acne severity, erythema, edema, blistering, abnormal pigmentation (hyper- or hypo-), and scarring.

The results demonstrate that in the regions receiving the treatment regime, there was a statistically significant decrease in the numbers of lesions in those regions (FIG. 8A). In contrast, in the regions receiving the control regime, there was no statistically significant decrease in the numbers of lesions in those regions (FIG. 8B).

Equivalents

While the invention has been particularly shown and described with reference to specific embodiments, it should be understood by those skilled in the art that various changes in form and detail may be made therein without departing from the spirit and scope of the invention as defined by the appended claims.

Incorporation By Reference

The content of each patent publication and scientific article identified hereinabove is expressly incorporated by reference herein.

What is claimed is:

1. A method of treating a sebaceous follicle disorder in a preselected region of mammalian skin, the preselected region having at least one lesion characteristic of the disorder disposed therein, the method comprising the steps of:
(a) cooling an exposed surface of the preselected region; and
(b) applying light having a wavelength in a range of from 0.95 microns to 1.16 microns, from 1.30 microns to 1.65 microns, or from 1.85 microns to 2.20 microns to the preselected region in an amount sufficient to ameliorate the lesion.

2. The method of claim 1, wherein in step (b) the light is laser light or incoherent light.

3. The method of claim 1, wherein in step (b) the light is laser light.

4. The method of claim 2, wherein the light has a wavelength in the range from 0.95 microns to 1.16 microns.

5. The method of claim 4, wherein the wavelength is in the range from 0.97 microns to 1.15 microns.

6. The method of claim 5, wherein the wavelength is in the range from 1.00 microns to 1.10 microns.

7. The method of claim 2, wherein the light has a wavelength in the range from 1.30 microns to 1.65 microns.

8. The method of claim 7, wherein the wavelength is in the range from 1.32 microns to 1.6 microns.

9. The method of claim 8, wherein the wavelength is in the range from 1.37 microns to 1.55 microns.

10. The method of claim 9, wherein the wavelength is in the range from 1.40 microns to 1.50 microns.

11. The method of claim 10, wherein the wavelength is 1.45 microns.

12. The method of claim 2, wherein the light has a wavelength in the range from 1.85 to 2.20 microns.

13. The method of claim 12, wherein the wavelength is in the range from 1.90 to 2.15 microns.

14. The method of claim 13, wherein the wavelength is in the range from 1.91 microns to 2.10 microns.

15. The method of claim 3, wherein the laser light comprises a fluence in the range from about 0.1 to about 500 joules per square centimeter.

16. The method of claim 15, wherein the fluence is in the range from about 5 to about 150 joules per square centimeter.

17. The method of claim 1, wherein step (a) occurs prior to step (b).

18. The method of claim 1 or 17, wherein step (a) occurs contemporaneously with step (b).

19. The method of claim 1, comprising the additional step of prior to step (b) providing a light absorbing material to the preselected region.

20. The method of claim 1, wherein in step (b) the thermal change occurs in the absence of an exogenously provided radiation absorbing material.

21. The method of claim 1, wherein the disorder is acne.

22. The method of claim 21, wherein the acne is acne vulgaris.

23. The method of claim 1 or 21, wherein applying light in step (b) reduces the size of a lesion disposed within the preselected region.

24. The method of claim 1 or 21, wherein applying light in step (b) reduces the density of lesions disposed within the preselected region.

25. The method of claim 1 or 21, wherein applying light in step (b) reduces lesion-associated skin inflammation in the preselected region.

26. A method of treating acne in a preselected region of mammalian skin, the preselected region having at least one acne lesion disposed therein, the method comprising the steps of:
(a) cooling an exposed surface of the preselected region; and
(b) exposing the preselected region light having a wavelength in the range from 1.3 microns to 1.65 microns to ameliorate the lesion.

27. The method of claim 26, wherein in step (b) the wavelength is in the range from 1.37 microns to 1.55 microns.

28. The method of claim 27, wherein the wavelength is 1.45 microns.

29. The method of claim 26, wherein in step (b) the light has a fluence in the range from about 0.1 to about 500 joules per square centimeter.

30. The method of claim 29, wherein the fluence is in the range from about 5 to about 150 joules per square centimeter.

31. The method of claim 26, wherein step (a) occurs prior to step (b).

32. The method of claim 26 or 31, wherein step (a) occurs contemporaneously with step (b).

33. The method of claim 26, wherein the disorder is acne vulgaris.

34. The method of claim 26, wherein applying light in step (b) reduces the size of a lesion disposed within the preselected region.

35. The method of claim 26, wherein applying light in step (b) reduces the density of lesions disposed within the preselected region.

36. The method of claim 26, wherein applying light in step (b) reduces lesion-associated skin-inflammation in the preselected region.
